(19) United States
(12) Patent Application Publication
(10) Pub. No.: US 2025/0205493 A1
(43) Pub. Date: Jun. 26, 2025

(54) NERVE STIMULATION DEVICE FOR CHRONIC PAIN MANAGEMENT

(71) Applicants: ROWAN UNIVERSITY, Glassboro, NJ (US); THE COOPER HEALTH SYSTEM, CAMDEN, NJ (US)

(72) Inventors: Wei XUE, Haddon Township, NJ (US); Jeffrey HETTINGER, Cherry Hill, NJ (US); Xiao HU, Cherry Hill, NJ (US); Robert HIRSH, Merion Station, PA (US)

(21) Appl. No.: 18/845,357

(22) PCT Filed: Mar. 9, 2023
(86) PCT No.: PCT/US23/14903

§ 371 Date: Sep. 9, 2024

Related U.S. Application Data
(60) Provisional application No. 63/318,242, filed on Mar. 9, 2022.

(51) Int. Cl. A61N 1/36; A61L 31/08; A61L 31/14; A61L 31/16; A61N 1/05; A61N 1/378

(52) U.S. Cl. CPC A61N 1/36071; A61L 31/088; A61L 31/146; A61L 31/16; A61N 1/0551; A61N 1/36153; A61N 1/36171; A61N 1/36175; A61N 1/3787; A61L 2300/102; A61L 2300/406

(57) ABSTRACT

A nerve stimulation device for delivering pain management electrical pulses to nerves includes a flexible substrate that is skin-mountable or implantable. An implantable lead includes microelectrodes provided with a coating for improving charge delivery via a porous electrode structure, and/or to reduce infection risk. In one embodiment, a flexible-substrate wearable energy transmitter patch transmits a power signal wirelessly (e.g., via induction coils) through the skin to an implanted flexible-substrate stimulator component that receives the power signal, generates a tonic signal for pain mitigation, and delivers the tonic signal to nerve stimulation points adjacent the electrodes of an implanted lead. In another embodiment, a flexible-substrate stimulator includes a power source and pulse generator, and the tonic signal is transmitted wirelessly through the skin to an implanted lead having electrodes for delivering the pulses to the stimulation points. Accordingly, the device can be worn without skin perforation, which reduces infection risk.

### CROSS-REFERENCE TO RELATED APPLICATION

This application claims the benefit of U.S. Provisional Patent Application No. 63/318,242 filed Mar. 9, 2022, the entire disclosure of which is hereby incorporated herein by reference.

### FIELD OF THE INVENTION

The present invention relates generally to nerve stimulation devices, and more particularly, to a nerve stimulation device that can be applied in a minimally invasive procedure, and that has increased biocompatibility, poses a reduced risk of infection, and is less obtrusive to wear.

### DISCUSSION OF RELATED ART

Chronic pain is prevalent for the general population, for a variety of reasons. One method of treatment of chronic pain is with opioids. The opioid epidemic has become one of the most devastating health crises in modern U.S. history. Recent statistical studies show that more than 72,000 Americans die each year from drug overdose and 11.4 million people misuse opioid drugs nationwide. The economic burden of opioid misuse is estimated as $78.5 billion per year, with $28.9 billion directly related to the healthcare and substance abuse treatment costs. As the long-term benefits (and detriments) of using opioids to treat chronic pain still need more rigorous assessment, other safe treatment options have become increasingly important.

One such option is spinal cord stimulation (SCS), which provides electrical stimulation to targeted areas in the nervous system. SCS has been proven clinically effective since the 1960s and is now considered a well-established method for treating chronic pain. A typical SCS system includes a compact stimulator to generate electrical pulses, and thin wires with small electrodes to deliver the electrical pulses to specific nerves on the spinal cord. The pulse patterns can mask pain signals travelling from nerves to the brain.

In one exemplary group of state-of-the-art SCS devices, the patient needs to undergo surgery to have both the stimulator and the leads implanted inside/under the body (under the skin), which requires a sizable incision to allow for insertion of the stimulator housing, etc. Such implantation of a large foreign body under the skin is associated with a risk of infection. State of the art SCS stimulators of this type generally include various components fixed to a rigid substrate (e.g., silicon-based printed circuit board) and/or placed in a rigid (e.g., hard plastic) housing, and supported externally to the body. These systems contain rechargeable batteries, which can be recharged using external charging belts and chargers. Although these state-of-the-art SCS stimulators are compact (e.g., Abbott's stimulator has dimensions of 5.55 cm×4.95 cm×1.34 cm), they still present physical and other challenges for patients, especially for those who have an active lifestyle.

In another exemplary group of state-of-the-art SCS devices, the SCS devices have a minimally invasive design in which the stimulator is worn on the outside surface of or otherwise outside the skin with only the leads implanted inside the body, by a small surgery being performed on the patient to insert the leads under the skin. However, these devices are still based on rigid-substrate (e.g., printed circuit board)/rigid-structure technology. For example, certain systems (e.g., Bioness StimRouter or Stimwave) use external stimulators mounted on conductive gel patches that are worn on the skin, but the overall device is rigid. However, the rigidness and bulkiness of these systems are still burdensome for patients who have to wear them every day.

What is needed is a nerve stimulation device capable of delivering electrical pulses to nerves (e.g., spinal cord or peripheral nerves) to manage pain that can be applied with a minimally invasive procedure, that reduces infection risk, improves specificity and that is less burdensome to wear. The present invention fulfills these needs, among others.

### SUMMARY

The present invention provides a nerve stimulation device that is capable of delivering electrical pulses to nerves to manage pain. The inventive nerve stimulation device can be applied with a minimally invasive procedure, reduces infection risk, can stimulate specific neurons or neuron clusters, and is relatively less burdensome to wear than current nerve stimulation (e.g., SCS) devices.

More particularly, the present invention provides a biocompatible nerve stimulation device that consists of two primary components: a stimulator (pulse generator) fabricated on a flexible substrate that can be either worn on the surface of the skin as an adhesive patch or be implanted as a flexible implant, and an implanted lead including microelectrodes or a microelectrode array (MEA) provided with electrochemically active coatings, which can improve specificity, allow for sufficient charge delivery with a relatively smaller lateral dimension of the electrode and smaller planar area/footprint, and reduce infection risk due to bactericidal properties of the coating. Accordingly, the stimulator of the nerve stimulation device can be worn on the surface of the skin without skin perforation, which reduces infection risk. Additionally, the implanted leads (and in some cases, the stimulator) can be applied with a minimally invasive surgical procedure, which also reduces infection risk. The implanted leads may be provided with coatings or other bactericidal elements to further reduce infection risk.

Further, the stimulator and the implanted leads may be fabricated on a flexible, biocompatible, and inexpensive protein-based substrate, which further reduces infection risk, and also makes the device less burdensome to wear than current SCS devices, or other rigid-substrate-based devices. More particularly, the stimulator skin patch may include a built-in pulse generating circuit fabricated on a flexible substrate to produce electrical signal patterns that can mask pain signals traveling from the nerves to the brain. The implantable electrical lead may contain microscale electrodes that can deliver the electrical signal patterns to the nerves at specific points.

Accordingly, the inventive biocompatible nerve stimulation device provides significant benefits, including: (1) the system is easy to use and relative less obtrusive; (2) patients have additional control over their health and budget due to the effectiveness and low-cost nature of the design; and (3) the risk of infection can be significantly reduced.

### BRIEF DESCRIPTION OF THE FIGURES

An understanding of the following description will be facilitated by reference to the attached drawings, in which:

FIG. 1 is a side view of a nerve stimulation device in accordance with an exemplary embodiment of the present invention, shown as applied to skin tissue;

FIG. 2 is a side view of a nerve stimulation device in accordance with an alternative embodiment of the present invention, shown as applied to skin tissue;

FIG. 3 is a schematic showing a simplified block diagram of an exemplary stimulator circuit in accordance with the exemplary embodiment of FIG. 1;

FIG. 4 is a top perspective view of an exemplary stimulator in accordance with the present invention; and

FIG. 5 is a bottom perspective view of the exemplary stimulator of FIG. 4, showing an exemplary electrode array on an exemplary lead.

### DETAILED DESCRIPTION

The present invention provides a nerve stimulation device 100 that is capable of delivering electrical pulses to nerves to manage pain. The inventive nerve stimulation device 100 can be applied with a minimally invasive procedure, reduces infection risk, can be made more biocompatible, can be used as a spinal cord stimulation device to stimulate spinal cord nerves and/or to stimulate peripheral nerves, and is relatively less burdensome to wear than current SCS devices.

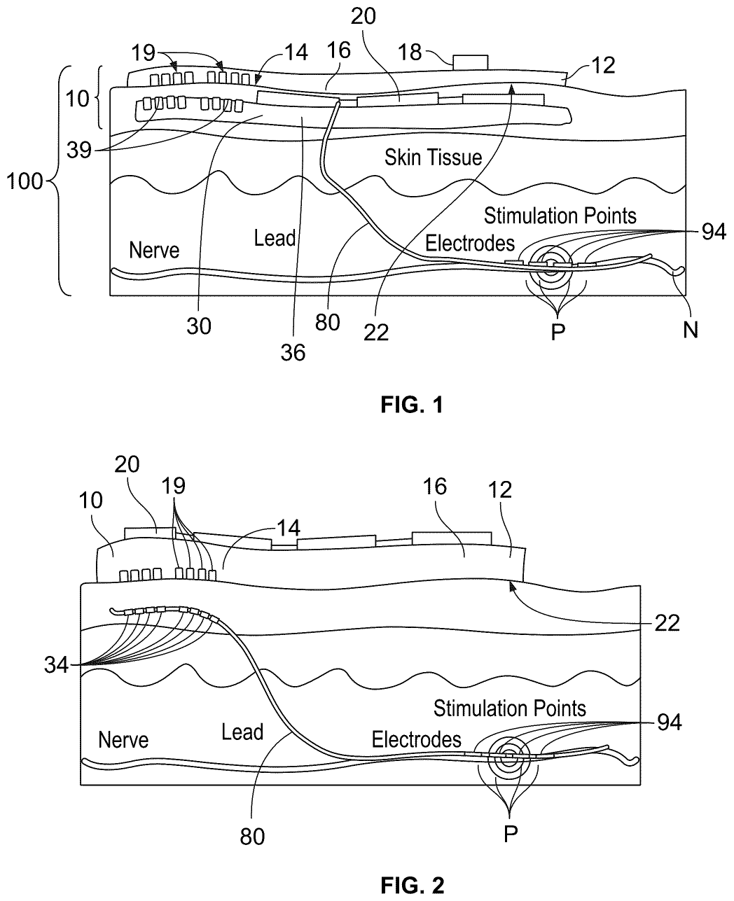

Referring now to FIGS. 1 and 2, a nerve stimulation device 100 in accordance with the present invention includes two primary components: a stimulator 10 and an implantable lead (or leads) 80. The stimulator 10 includes a pulse generating circuit 20 configured to generate electrical pulses suitable for delivery to bodily nerves for pain management purposes. Such pulse generators and suitable electrical pulses for pain management purposes are well-known in the art of SCS devices and are beyond the scope of the present invention, and thus are not discussed in greater detail herein. By way of example, tonic pulse train of repeating tonic pulses having a 100 μs pulse width and a frequency of 400 Hz (or intervals of 2.5 ms) and voltage of 3.7 V, may be suitable for this purpose. Any suitable pulse generating circuit 20 and electrical pulses may be used in accordance with the present invention. The stimulator 10 includes circuitry fabricated on a flexible substrate, which may be a polymer- or protein-based substrate. By way of example, suitable polymer-based substrate materials include polydimethylsiloxane (PDMS) and Kapton polyimide (PI) film, as they are flexible, chemically inert, and biocompatible and thus suitable for implantation in the body. This allows the stimulator to have a relatively thinner, lighter, and more flexible structure that is body-conforming and less obtrusive to wear. By way of further example, suitable protein-based substrate materials include silk protein-based materials such as Mori silk, Eri silk, Muga silk, and Tussah silk produced by different species of silkworms.

The implantable lead 80 comprises conductive electrical conductors (material) 90 encapsulated in non-conductive material 92 and exposed conductive electrodes 94 operatively connected to the pulse generating circuit 20 of the stimulator 10. The lead 80 is elongated and configured to be positionable adjacent bodily nerves to deliver electrical pulses generated by the pulse generating circuit 20 for pain management purposes. The implantable lead 80 is fabricated on a flexible substrate 82. Since the implantable lead 80 is to be implanted in bodily tissue, the flexible substrate 82 is preferably constructed of a biocompatible material, such as a protein-based material. By way of example, PDMS and PI film may be used as the material for the flexible substrate. This allows the device to have a relatively thinner, lighter, and more flexible structure that is body-conforming and less obtrusive to wear. In certain embodiments, flexible protein material that can be absorbed by the human body may be used, to allow for tuning of the implanted device's lifetime.

By way of example, the circuitry may be fabricated by depositing metals on the polymer and/or protein substrates by a well-known magnetron sputtering process (a physical vapor deposition (PVD) process). An exemplary configuration of a magnetic sputtering system consists of the source material (the material to be sputtered) and the substrate (the polymer to be coated) in a vacuum chamber facing parallel to each other. The system is evacuated to a low base pressure that removes any unwanted impurities such as water, oxygen, nitrogen, etc. Normally for pure metals, a DC source of >300 V is supplied to the source material which is the negatively charged cathode while the substrate is the positively charged anode (or electrically grounded, or floating). Then an inert gas is introduced to the system—typically argon, which becomes ionized in the presence of the large electric fields either applied or induced by trapping electrons in a magnetic field used in magnetron sputtering. The ionized argon particles bombard the surface of the source material knocking off the atoms in a vapor fashion only to condense on to the surface of the substrate. Magnetron sputtering has become a commonly used deposition technique in manufacturing surface coatings for industrial and research purposes due to the level of control it can offer users over power density, sputter pressure, and sputter time. By varying these parameters, coatings with a suitable surface structure, roughness, grain size, level of adhesion, etc. By way of example, deposited metals may be silver (Ag), copper (Cu), titanium (Ti), gold (Au), platinum (Pt) as the underlying electrode/conductor with a titanium nitride (TiN) coating (or TiN+ bactericidal element surface coating) to enhance charge exchange. The nitride surface coating is best applied using reactive magnetron sputtering in an argon and nitrogen gas mixture.

By way of example, known photolithography and stencil lithography techniques may be used to fabricate the necessary circuitry on the flexible substrates. Adhesion of coatings can be improved by providing a buffer layer coating, such as SiO₂.

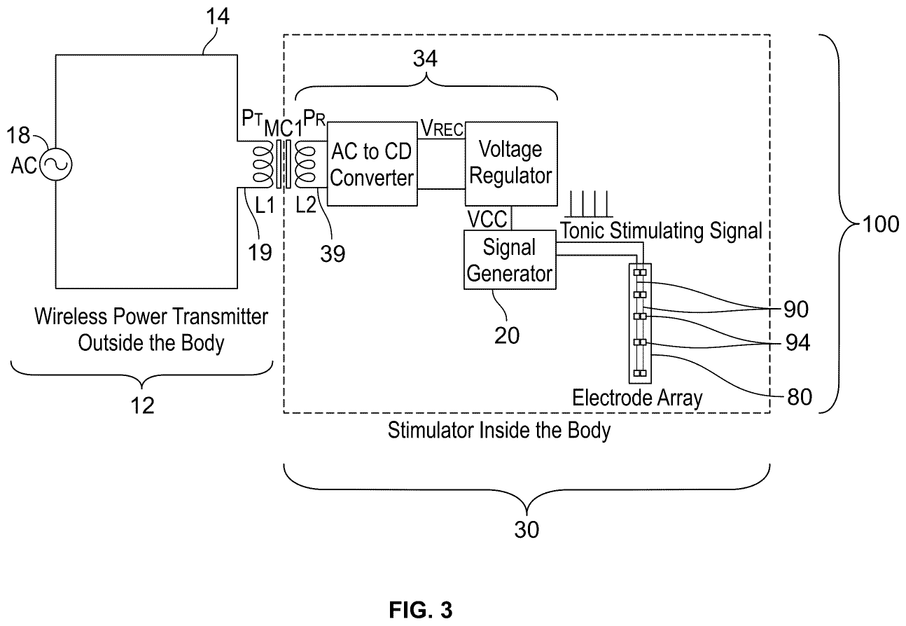

In the exemplary embodiment shown in FIGS. 1 and 3, the stimulator 10 comprises a transmitter patch 12 comprising transmitter circuitry 14 fabricated on a first flexible substrate 16. The transmitter circuitry 14 includes a power source 18 (such as a rechargeable battery) and traces or other structures serving as an energy transmitter antenna 19, e.g., for transmitting RF energy. A contact surface 22 of the substrate 16 bears adhesive for securing the transmitted patch 12 directly to an outer surface of the skin.

Further, this exemplary stimulator 10 comprises an implantable receiver 30 comprising receiver circuitry 34 fabricated on a second flexible substrate 36 suitable for implantation in the skin/under the surface of the skin. Accordingly, at least the implantable receiver 30 may be fabricated on a substrate 36 made of a biocompatible material, such as a protein-based substrate. The receiver circuitry 34 (which may be just specially-configured traces) forms an energy receiver antenna 39 (such as an induction coil) suitable for receiving energy transmitted by the transmitter circuitry 14.

Accordingly, the implantable receiver 30 of the device 100 does not include a battery, due to safety and lifetime limitations. Rather, a wireless power transmitter is located outside the body and is used to provide an operating voltage to the stimulator via inductive coupling. In inductive coupling, RF antennas are provided as coils configured such that a change in current through the primary coil induces a voltage in the secondary coil. The primary and secondary coils are thus transmitter and receiver coils in this case. The received alternating voltage is rectified by using a full bridge rectifier and converted into a DC voltage. This DC voltage may then be regulated by a voltage regulator.

By way of example, a power signal with an amplitude of 10 V and a frequency of 2.5 MHz may be transmitted wirelessly using inductive coils having dimensions of 7.87 inches×4.13 inches×0.2 inches (or considerably smaller, after manufacturing improvements) may be used to transmit a maximum wireless power of 2.5 W, which is sufficient for operating an exemplary stimulator. This enables the implantable portion of the nerve stimulation device 100 to become completely passive (battery-free), thereby eliminating a major concern (infection, chemical leakage and additional surgery-induced risk associated with internal batteries) for implantable biomedical devices.

By way of example, LM317 is a suitable exemplary commercially-available adjustable voltage regulator that is configured to provide a consistent and suitable 3.7 V supply voltage for the pulse generating circuit 20.

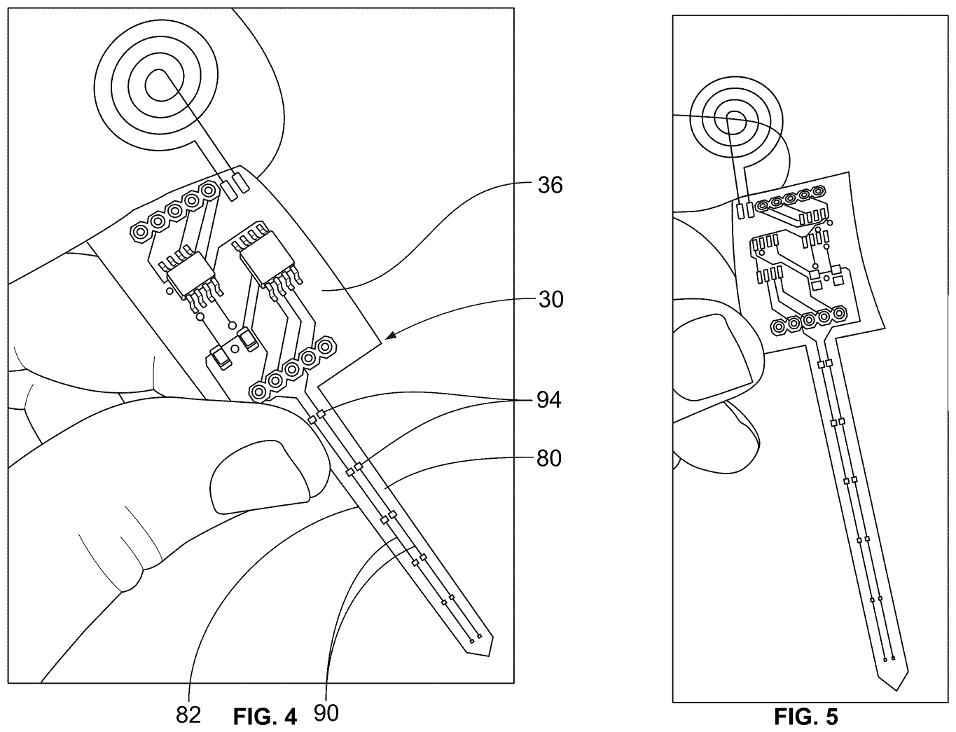

In this exemplary embodiment, the implantable receiver 30 includes the pulse generating circuit 20, which is operatively connected by conductive traces of the second flexible substrate 36 to the receiver circuitry 34 (including the induction coil) of the implantable receiver, as best shown in FIG. 4. The pulse generating circuit 20 includes circuitry fabricated on a flexible substrate.

Accordingly, energy supplied by the power source 18 of the externally-worn transmitter patch 12 is transmitted by the transmitter circuitry 14 of the transmitter patch, through the surface of the skin, and is received by the antenna of the receiver circuitry 34 of the implantable receiver 30 implanted beneath the circuity of the skin. The transmitted energy received by the implantable receiver 30 powers the pulse generating circuit 20, which in turn generates electrical pulses suitable for pain management. The pulses are delivered to suitable nerves via the leads 80 conductively coupled to the pulse generating circuit 20 and associated conductive electrodes 94 positioned at stimulation points P along bodily nerves N. The leads 80 and electrodes 94 may be implanted in suitable locations by a minimally invasive surgical operation that involves skin incision and lead insertion procedures as well known in the art. Suitable implantation methods are known in the field of spinal cord stimulators, for example, and are thus not discussed in greater detail herein. Any suitable implantation method may be used in accordance with the present invention.

Accordingly, in this embodiment, both the stimulator 10 and the leads 80 are implanted inside the body, beneath the surface of the skin, as shown in FIG. 1. The device 100 (including the transmitter patch 12 and implantable receiver 30) is fabricated using flexible substrates, and thus is flexible and has an ultrathin profile, making it less obtrusive and burdensome as compared to prior art implanted spinal cord stimulation and/or other nerve stimulation devices. The device 100 is powered through an embedded RF energy receiver, which obtains its energy from an externally-worn transmitter patch worn directly on the skin.

In other embodiments, the transmitter patch 12 may not be worn directly on the skin, but rather may be carried adjacent the skin, e.g., by a suitable belt or other apparatus.

In the alternative exemplary embodiment shown in FIG. 2, the stimulator 10 comprises a transmitter patch 12 comprising transmitter circuitry 14 fabricated on a flexible substrate 16 (e.g., a flexible polymer or protein substrate). The transmitter circuitry 14 includes a power source 18 (such as a rechargeable battery) and traces or other structures serving as an energy transmitter antenna 19, e.g., for transmitting RF energy. A contact surface 22 of the substrate 16 bears adhesive for securing the transmitted patch 12 directly to an outer surface of the skin.

Unlike the embodiment of FIG. 1, this exemplary stimulator 10 does not include an implantable receiver. Instead, the pulse generating circuit 20 is supported on the flexible substrate 16. The pulse generating circuit 20 includes circuitry fabricated on a flexible substrate.

In this embodiment, only the leads 80 are implanted beneath the surface of the skin. Since the implantable lead 80 is to be implanted in bodily tissue, the flexible substrate 82 is preferably constructed of a biocompatible material, such as a protein-based material. In this embodiment, the lead 80 further include receiver circuitry 34 (which may be just specially-configured traces) forming a receiver antenna suitable for receiving (e.g., by inductive coupling) energy transmitted by the transmitter antenna 19 of the transmitter circuitry 14.

Accordingly, energy supplied by the power source 18 of the externally-worn transmitter patch 12 powers the pulse generating circuitry 20, which in turn generates electrical pulses suitable for pain management. The pulses are transmitted by the transmitter antenna 19 of the transmitter circuitry 14 on the externally-worn transmitter patch 12, through the surface of the skin, and are received by the receiver antenna of the receiver circuitry 34 of the lead 80 implanted beneath the surface of the skin. The pulses are delivered to suitable nerves via the lead 80 conductively coupled to the associated conductive electrodes 94 positioned at stimulation points P along bodily nerves N. The leads 80 and electrodes 94 may be implanted in suitable locations by a minimally invasive surgical operation that involve skin incision and lead insertion procedures as well known in the art. Suitable implantation methods are known in the field of spinal cord stimulators, and are thus not discussed in greater detail herein. Any suitable implantation method may be used in accordance with the present invention.

Accordingly, in this embodiment, only the lead 80 is implanted inside the body, beneath the surface of the skin, as shown in FIG. 2. The device 100 (including the transmitter patch 12 and the lead 80) is fabricated using flexible substrates, and thus is flexible and has an ultrathin profile, making it less obtrusive and burdensome as compared to prior art implanted SCS devices. The device 100 is powered by an external battery, and generated pulses are transmitted through an external RF energy transmitter 14 to an embedded RF energy receiver 34 through inductive coupling, then to the lead 80.

Accordingly, the stimulator 10 of the nerve stimulation device 100 can be worn on the surface of the skin, which reduces infection risk. Additionally, the implanted lead 80 (and in some cases, at least a portion of the stimulator) can be applied with a minimally invasive surgical procedure, which also reduces infection risk. Additionally, applied coatings further reduce infection risk. Further, the stimulator and the implanted lead are fabricated on a flexible substrate, which may be a biocompatible protein-based substrate, which further reduces infection risk, and also makes the device less burdensome to wear than current SCS devices. More particularly, the stimulator skin patch includes a built-in pulse generating circuit fabricated on a flexible substrate to produce electrical signal patterns that can mask pain signals traveling from the nerves to the brain. The implantable electrical lead contains microscale electrodes that can deliver the electrical signal patterns to the nerves at specific points, as desired.

Referring again to FIG. 3, a simplified block diagram of the nerve stimulation device of FIG. 1 is shown. As will be appreciated from FIG. 3, the nerve stimulation device includes pulse generating circuit 20 as part of the stimulator 10, an electrode array as part of the lead 80, and a wireless power receiving module as part of the stimulator. For FIG. 2, the external nerve stimulation device includes the pulse generating circuit 20 and wireless power transmission module 14 as part of the stimulator. The implanted lead 80 includes wireless power receiver module 34 and electrode array 94. The pulse generating circuit 20 produces the Tonic Stimulating Pulses and the electrodes are used to provide these pulses to the spinal cord or peripheral nerves of the human body. The power receiving module is responsible for receiving power wirelessly, thus making the implantable portion of the device completely passive.

The pulse generating circuit 20 is designed to produce and control the amplitude of the Tonic pulses. By way of example, an ATtiny85 microcontroller may be programmed to generate stimulating pulses. As known in the art, the ATtiny85 microcontroller is a standalone chip that consists of an 8-bit ADC and an 8 MHz microcontroller. The chip has 5 I/Os (input/output) which can be used for both analog and digital purposes. The chip may be programmed using Arduino as ISP (In-System Programmer). The microcontroller may be programmed to generate tonic pulses with the following specifications: pulse width 100 μs and pulse frequency of 400 Hz. In order to control the amplitude of the signal, a potentiometer, such as an X9C104 digitally programmable potentiometer providing a variable resistance ranging from 40 Ω to 100 kΩ, may be used. The output of the pulse generating circuit may be fed into the potentiometer-based voltage divider configuration, which can produce tonic pulses with amplitude ranging from 320 mV to 3.7 V. The output of the pulse generating circuit may be connected with a load variable resistor, such that the resistance may be varied from 1 kΩ to 25 Ω, and associated output voltage may be provided with maximum output power of 180 mW which is sufficient to be used for implantable applications.

Referring now to FIGS. 4 and 5, top and bottom perspective views of an exemplary stimulator 10 and lead 80 are shown. As will be appreciated from FIGS. 4 and 6, the lead 80 includes a pair of spaced conductive traces having exposed electrodes 94. In the example shown, each electrode in the electrode pair is square in shape and has a size measuring one of 1.0 mm 0.8 mm, 0.6 mm, 0.5 mm, 0.4 mm and 0.3 mm for illustrative purposes.

The pulse generating circuit 20 is interfaced with the spinal cord of the human by an electrode array of the lead 80. Electrodes with a bigger surface area provide lower contact impedance and hence an easier flow of stimulating signal through the spinal cord. Meanwhile smaller electrodes provide higher resolution, which can lead to higher specificity for pain management because individual neurons can now be targeted. The area of the electrodes 94 may be varied as desired to achieve a desired effect in delivering the generated pulses to the spinal cord or peripheral nerves.

Small electrodes are generally desired, to suppress the body's immuno-response, which tends to isolate objects implanted in the body. This response has been shown to be related to the size of the implanted object. Thus, smaller electrodes would reduce this response, cause less trauma during implantation, and provide better specificity possibly activating individual neurons. Providing a relatively small electrode with a suitable coating can increase the effective charge exchange of the electrode, to mimic the performance of a relatively larger electrode without actually increasing its lateral physical size/dimensions, to minimize the immuno-response.

In an exemplary embodiment, the electrodes are provided with a biocompatible coating for the purpose of increasing the effective charge exchange of a relatively small electrode. For example, a biocompatible titanium nitride (TiN) material that is reactively sputtered to form pillars with extended/correlated pores between the pillars is a suitable coating. Other stable and inert coatings, such as zirconium nitride (ZrN), with similar microstructures may be used. These pores (voids between pillars) are accessible by the ion-containing biological solutions (phosphate buffered saline-PBS which mimics blood plasma), effectively increasing the surface area of the electronics. When deposited using specific deposition conditions, the coatings result in a series of conducting pillars with a large effective surface area.

In certain embodiments, powers in the range of about 130 W to about 225 W, deposition pressure of about 5-20 milliTorr (e.g., 10 milliTorr), and nitrogen partial pressure of about 5%-40% (e.g., 10%), with an overall thickness in excess of about 1-16 μm (e.g., 2 μm), have been found suitable for many nerve stimulation applications. The bactericidal elements may be deposited at much lower powers (e.g., about 10 W) due to the ease of sputtering most of these metals and the desired quantity.

By way of example, bactericidal elements can be mixed with the (TiN or other) coatings without detrimentally impacting the charge exchange performance of the coatings to reduce the probability of infection. In addition, the porous nature of the coatings (pores between pillars) can be loosely loaded with an antibiotic material. In addition to adding coatings for enhanced charge exchange, coatings may be added, in accordance with the present invention, to increase bactericidal properties of the implanted portion of the device 100, and to thereby reduce infection risk. In one embodiment, pores of a TiN coating of the lead 80 are loaded with an antibiotic, which will then release over time following implantation to reduce infection risk By way of example, the pores may be loaded with an antibiotic by spraying or dipping of the lead 80. By way of example, suitable antibiotics include penicillin and its derivatives, such as cloxacillin, glycopeptides such as teicoplanin and aminoglycosides such as gentamicin.

Alternatively, elements having bactericidal properties, such as Zn, Ag, and Cu, may be added to the charge exchange enhancing coating to suppress for formation of biofilm on the electrode. In addition, when in use as an electrode, the bactericidal element oxidizes and is released. The resulting ions are bactericidal and kill distal bacteria in solution. For example, added elements having a concentration in the range of about 1% to about 15% may be suitable. By combining bactericidal elements with high-performing TiN coatings it is possible to suppress infections associated with implanted spinal cord stimulation electrodes. The bactericidal elements prevent biofilm formation (bacteria colonization on the electrodes) without an added potential. When a potential is applied, the bactericidal ions undergo oxidation and are released allowing the killing of distal bacteria. This is useful short term after the implantation procedure or long-term for more permanently implanted devices.

In use, the lead may be implanted using a conventional surgical procedure, to position the electrodes of the lead adjacent nerve stimulation points. In certain embodiments, the energy receiver antenna of the lead 80 and/or flexible-substrate stimulator 30 may also be implanted beneath the surface of the skin in a surgical procedure, with the energy-receiving antenna positioned near the surface of the skin. A release sheet may be removed from a flexible-substrate of a wearable stimulator 10 and/or wearable energy transmitter patch 12 to expose adhesive. The stimulator 10 or energy transmitter patch 12 may then be positioned on and be adhered to an outer surface of the skin, in a location in which the energy transmitter antenna 14 positioned adjacent the energy receiver antenna 39 beneath the surface of the skin. Operation of the device thereby causes transmission of a power signal or a tonic signal through the skin to the implanted portion of the device, with the result of the electrical pulses of the tonic signal being delivered to stimulation points adjacent the relevant nerve via the implanted lead. In the event of a need for replacement of a battery, the external portion of the device may be changed (or the batter may be changed) without the need for an incision to access the implanted portion of the device.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A nerve stimulation device comprising:
a flexible stimulator comprising circuitry supported on a flexible substrate, said stimulator comprising:
a wearable energy transmitter patch, said energy transmitter patch comprising:
a first flexible substrate;
a power source supported on said first flexible substrate; and
transmitter circuitry electrically coupled to said power source and supported on said first flexible substrate, said transmitter circuitry defining an energy transmitter antenna configured to transmit a power signal wirelessly from said power source; and
an implantable receiver, said implantable receiver comprising:
a second flexible substrate;
receiver circuitry defining an energy receiver antenna configured to receive the power signal transmitted from said wearable energy transmitter patch by said energy transmitter antenna;
pulse generator circuitry operatively connected to said receiver circuitry to receive the power signal and generate electrical pulses operative to mask pain signals from a nerve;
an implantable lead comprising:
a first portion electrically coupled to said pulse generator circuitry to receive said electrical pulses; and
a second portion electrically coupled to said first portion and comprising at least one electrode positionable adjacent the nerve to deliver the electrical pulses to the nerve and thereby mask pain signals from the nerve.

2. The nerve stimulation device of claim 1, wherein at least one of said first flexible substrate and said second flexible substrate is constructed of a polymer material.

3. The nerve stimulation device of claim 1, wherein at least one of said first flexible substrate and said second flexible substrate is constructed of a protein-based material.

4. The nerve stimulation device of claim 1, wherein the wearable energy transmitter patch defines a contact surface, and wherein the contact surface is provided with a skin-compatible adhesive.

5. The nerve stimulation device of claim 1, wherein said energy transmitter antenna and said energy receiver antenna are configured as RF antennas.

6. The nerve stimulation device of claim 1, wherein said energy transmitter antenna and said energy receiver antenna are configured as inductive coils.

7. The nerve stimulation device of claim 1, wherein said energy transmitter antenna is configured to provide an operating voltage to said energy receiver antenna via inductive coupling.

8. The nerve stimulation device of claim 1, wherein said pulse generator circuitry is configured to generate electrical pulses operative to mask pain signals from a nerve in the form of a pulse train of tonic pulses having at least one of a pulse width of about 100 μs and a pulse frequency of about 400 Hz, and an amplitude in the range of about 320 mV to 3.7 V.

9. The nerve stimulation device of claim 1, wherein said pulse generator circuitry is configured to supply tonic pulses to a load variable resistor having a resistance variable from about 1 kΩ to about 25 Ω, to provide an associated output voltage of up to about 180 mW.

10. The nerve stimulation device of claim 1, wherein said at least one electrode comprises one of a microelectrode and a microelectrode array (MEA) provided with an electrochemically active coating operable to increase charge delivery via said at least one electrode.

11. The nerve stimulation device of claim 1, wherein said at least one electrode comprises one of a microelectrode and a microelectrode array (MEA) provided with one of a bactericidal coating and a bactericidal element.

12. The nerve stimulation device of claim 11, wherein said the bactericidal coating comprises one of titanium nitride and zirconium nitride.

13. The nerve stimulation device of claim 11, wherein said bactericidal coating is constructed of a material sputtered to form pillars defining pores therebetween.

14. The nerve stimulation device of claim 13, wherein said bactericidal coating further comprises an antibiotic material disposed in the pores.

15. The nerve stimulation device of claim 1, wherein said bactericidal element is selected from a group consisting of zinc, silver, gold, platinum and copper.

16. The nerve stimulation device of claim 1, wherein said implantable receiver comprises a full bridge rectifier for converting received alternating voltage to DC voltage.

17. The nerve stimulation device of claim 16, wherein said implantable receiver further comprises a voltage regulator operable to regulate DC voltage.

18. A nerve stimulation device comprising:
a flexible stimulator comprising circuitry supported on a flexible substrate, said stimulator comprising:
a flexible substrate;
a power source supported on said flexible substrate;
pulse generator circuitry operatively connected to said power source to receive a power signal and generate electrical pulses operative to mask pain signals from a nerve; and
transmitter circuitry electrically coupled to said pulse generator circuit, said transmitter circuitry defining an energy transmitter antenna configured to transmit a pulse signal wirelessly from said stimulator; and
an implantable lead comprising:
receiver circuitry defining an energy receiver antenna configured to receive the inductively coupled signal transmitted from said stimulator by said energy transmitter antenna;
a first portion electrically coupled to said receiver circuitry to receive said electrical pulses; and
a second portion electrically coupled to said first portion and comprising at least one electrode positionable adjacent the nerve to deliver the electrical pulses to the nerve and thereby mask pain signals from the nerve.

19. The nerve stimulation device of claim 18, wherein said first flexible substrate is constructed of a polymer material.

20. The nerve stimulation device of claim 18, wherein said flexible substrate is constructed of a protein-based material.

21. The nerve stimulation device of claim 18, wherein said stimulator defines a contact surface, and wherein said contact surface is provided with a skin-compatible adhesive.

22. The nerve stimulation device of claim 18, wherein said energy transmitter antenna and said energy receiver antenna are configured as RF antennas.

23. The nerve stimulation device of claim 18, wherein said energy transmitter antenna and said energy receiver antenna are configured as inductive coils.

24. The nerve stimulation device of claim 18, wherein said energy transmitter antenna is configured to provide a pulse train of tonic pulses to said energy receiver antenna via inductive coupling.

25. The nerve stimulation device of claim 18, wherein said pulse generator circuitry is configured to generate electrical pulses operative to mask pain signals from a nerve in the form of a pulse train of tonic pulses having at least one of a pulse width of about 100 μs and a pulse frequency of about 400 Hz, and an amplitude in the range of about 320 mV to 3.7 V.

26. The nerve stimulation device of claim 18, wherein said pulse generator circuitry is configured to supply tonic pulses to a load variable resistor having a resistance variable from about 1 kΩ to about 25 Ω, to provide an associated output voltage of up to about 180 mW.

27. The nerve stimulation device of claim 18, wherein said at least one electrode comprises one of a microelectrode and a microelectrode array (MEA) provided with an electrochemically active coating operable to increase charge delivery via said at least one electrode.

28. The nerve stimulation device of claim 18, wherein said at least one electrode comprises one of a microelectrode and a microelectrode array (MEA) provided with one of a bactericidal coating and a bactericidal element.

29. The nerve stimulation device of claim 28, wherein said the bactericidal coating comprises one of titanium nitride and zirconium nitride.

30. The nerve stimulation device of claim 28, wherein said bactericidal coating is constructed of a material sputtered to form pillars defining pores therebetween.

31. The nerve stimulation device of claim 30, wherein said bactericidal coating further comprises an antibiotic material disposed in the pores.

32. The nerve stimulation device of claim 18, wherein said bactericidal element is selected from a group consisting of zinc, silver, gold, platinum and copper.

33. The nerve stimulation device of claim 18, wherein said receiver circuitry comprises a full bridge rectifier for converting received alternating voltage to DC voltage.

34. The nerve stimulation device of claim 33, wherein said receiver circuitry further comprises a voltage regulator operable to regulate DC voltage.

35. A nerve stimulation device comprising:
a flexible substrate;
transmitter circuitry electrically coupled to a power source and supported on said flexible substrate, said transmitter circuitry defining an energy transmitter antenna configured to transmit a signal wirelessly;
pulse generator circuitry operatively connected to receive a power signal and generate electrical pulses operative to mask pain signals from a nerve;
an implantable energy receiver antenna configured to receive the signal transmitted from said energy transmitter antenna; and
an implantable lead electrically coupled to said pulse generator circuitry to receive said electrical pulses, and comprising at least one electrode positionable adjacent the nerve to deliver said electrical pulses to the nerve and thereby mask pain signals from the nerve.
